(19) United States
(12) Patent Application Publication
(10) Pub. No.: US 2010/0311691 A1
(43) Pub. Date: Dec. 9, 2010

(54) COMPOSITIONS OF S-ALKYLISOTHIOURONIUM DERIVATIVES FOR TREATING UPPER RESPIRATORY CONGESTION

(76) Inventors: Refael Barkan, Rishon Le Zion (IL); Victor Ghicavii, Chisinau (MD)

Correspondence Address: BROWDY AND NEIMARK, P.L.L.C.;624 NINTH STREET, NW, SUITE 300, WASHINGTON, DC, 20001-5303, US

(21) Appl. No.: 12/741,730

(22) PCT Filed: Nov. 6, 2008
(86) PCT No.: PCT/IL08/01465

§ 371 Date: Aug. 18, 2010

(30) Foreign Application Priority Data
Nov. 6, 2007 (MD) A 2007 0301

(51) Int. Cl. A61K 31/661; A61P 11/14; A61P 11/10; A61P 11/12; A61P 11/02

(52) U.S. Cl. 514/76

(57) ABSTRACT

The present invention provides intranasal pharmaceutical compositions comprising an S-alkylisothiouronium derivative and methods of treating or alleviating upper respiratory and oral-pharyngeal congestions by pharmaceutical compositions comprising the S-alkylisothiouronium derivative.

### FIELD OF THE INVENTION

The present invention provides intranasal pharmaceutical compositions comprising an S-alkylisothiouronium derivative and methods of treating or alleviating upper respiratory and oral-pharyngeal congestions by pharmaceutical compositions comprising the S-alkylisothiouronium derivative.

### BACKGROUND OF THE INVENTION

People around the world frequently suffer from upper respiratory tract and oral pharyngeal congestion. The upper respiratory tract and oral pharyngeal congestion can be caused by infections in the respiratory tract and/or oral and pharyngeal cavities, allergies, changes in weather conditions, as well as from the overall health and genetic disposition of the person. Generally the congestion is diagnosed as partially or fully blocked air passages including airways in the nose, mouth, throat, and lungs. Other symptoms related to the cause typically accompany the congestion. Cold symptoms such as fever, runny nose, sneezing, loss of taste and smell, sinus infections, cough, tickles in the throat, sinus pain, headache, and throat or gland pain are some of the more common symptoms found with upper respiratory and oral-pharyngeal congestion.

Congestion of the upper respiratory tract and oral pharyngeal cavity and related symptoms generally have undesirable effects for the afflicted person. For example, the congestion may affect performance in the workplace, school, and at home up to and including loss of work and loss of school attendance. Further, congestion may reduce the ability to perform routine activities, such as housework, driving, running errands, and may even totally incapacitate the person. Severe and intolerable congestion often requires visits to the hospital and treatment. In addition, viral or bacterial infections of the sinus passage or other airways may be passed to healthy persons through symptoms of the congestion. For example, a cough or sneeze may convey a bacterium or virus to another person. Thus, upper respiratory tract and oral pharyngeal congestion and its symptoms need to be treated.

Generally, there are two typical approaches to treating symptoms of the congestion. One approach involves initially treating the underlying cause of the symptom. For example, a bacterial infection is generally treated by administering an antibiotic to kill the bacteria causing the infection. The second approach involves treating the symptoms themselves, typically in addition to treating the underlying cause, by independently administering one or more medications for relief of specific symptoms. For example, an antitussive agent, commonly referred to as a cough suppressant, has been typically administered for the treatment or relief of cough. An opioid medication, such as codeine and hydrocodone bitartrate, has generally been administered to relieve pain consistent with the congestion while suppressing a cough. Also decongestants, such as phenylephrine and pseudoephedrine, have been administered to patients for reducing mucosal swelling and draining the mucus build-up to clear congestion in the air passages. Symptoms due to allergies or allergens are often treated with an antihistamine. Antihistamines, often referred to as histamine-class receptor blockers, are compounds that may antagonistically block the histamine receptor from binding histamine thereby preventing the symptoms of an allergy. Examples of antihistamines include brompheneramine maleate, chlorpheneramine maleate, and diphenhydramine, all of which have shown good clinical efficacy.

U.S. Pat. No. 7,148,208 to Barkan et al. discloses methods for treating headaches and migraines as well as nausea and vomiting comprising administering to a subject in need of such treatments a composition comprising an S-alkylisothiouronium derivative.

International Application Publication No. WO 2007/029255 to some of the inventors of the present invention discloses methods for preventing hypotension and stabilizing blood pressure in hemodialysis patients comprising administering to these patients a pharmaceutical composition comprising an S-alkylisothiouronium derivative.

International Application Publication No. WO 2007/108004 to the inventors of the present invention discloses extended release dosage forms of S-alkylisothiouronium derivatives and methods of use thereof for treating chronic inflammation comprising administering to a subject in need of such treatment a pharmaceutical composition comprising an S-alkylisothiouronium derivative.

Recently in unpublished studies S-ethylisothiouronium bromide has been used to treat nasal congestion. However, S-ethylisothiouronium bromide was found to exert moderate and short-term nasal decongestions with harmful side effects.

There is still a need for improved methods for treating upper respiratory tract and oral pharyngeal congestions having fewer or no side effects.

### SUMMARY OF THE INVENTION

The present invention provides methods for treating or alleviating upper respiratory tract and oral pharyngeal congestion and symptoms associated therewith comprising administering to a subject in need of such treatment a pharmaceutical composition comprising an S-alkylisothiouronium derivative.

It is now disclosed that intranasal administration of a pharmaceutical composition comprising S-ethylisothiouronium diethylphosphate as the active agent to subjects suffering from upper respiratory tract and oral pharyngeal congestions and symptoms associated therewith resulted in an amelioration of the congestions and symptoms associated therewith at a shorter period of time than obtained by the commonly used medications.

Surprisingly, it is now disclosed that S-ethylisothiouronium diethylphosphate was more efficient than S-ethylisothiouronium bromide in alleviating upper respiratory tract and oral pharyngeal congestion and symptoms associated therewith. The decongestant effect of S-ethylisothiouronium diethylphosphate was achieved earlier than that obtained by S-ethylisothiouronium bromide, was longer-lasting, required fewer applications, and had little or no side effects.

According to a first aspect, the present invention provides an intranasal pharmaceutical composition of an S-alkylisothiouronium derivative comprising as an active agent a compound of formula I:

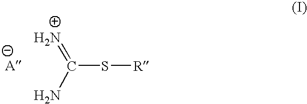

wherein

R″ is a straight or branched alkyl, optionally substituted by halogen; and

A″ (−) is an anion derived from a phosphorous containing acid, a phosphorous acid ester and a phosphorous acid amide; and wherein the compound of formula I is present in the pharmaceutical composition in an amount of from about 0.5% (w/w) to about 5% (w/w), further comprising a pharmaceutically acceptable carrier.

According to some embodiments, the anion of the compound of formula I is derived from a mono or di-alkyl ester of a phosphate or phosphite.

According to additional embodiments, the compound within the intranasal pharmaceutical composition is selected from the group consisting of:
S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

According to a certain embodiment, the compound within the intranasal pharmaceutical composition is S-ethylisothiouronium diethylphosphate.

According to some embodiments, the compound of formula I is present in the pharmaceutical composition in an amount of from about 1% (w/w) to about 3% (w/w). According to an exemplary embodiment, the compound of formula I is present in the pharmaceutical composition in an amount of about 2% (w/w).

According to further embodiments, the intranasal pharmaceutical composition is formulated in a form selected from the group consisting of a solution, suspension, spray, cream, gel, and ointment. According to an exemplary embodiment, the intranasal pharmaceutical composition is formulated as a solution.

According to yet further embodiments, the intranasal pharmaceutical composition further comprising at least one ingredient selected from the group consisting of buffers, isotonizing agents, preservatives, pH adjustors, thickeners, chelating agents, suspending agents, perfumes, and natural or synthetic plant extracts.

According to further embodiments, if the pharmaceutical composition is formulated as a solution, the solution has a pH of about 4 to about 7, preferably a pH of about 6 to about 7, more preferably the pharmaceutical composition has a pH of about 6.5.

According to yet further embodiments, the natural or synthetic plant extracts are aromatic oils. According to certain embodiments, the aromatic oil is selected from the group consisting of peppermint oil, eucalyptus oil, menthol, white fir tree oil, and wintergreen oil.

According to some embodiments, the intranasal pharmaceutical composition comprises about 0.5% (w/w) to about 5% (w/w) S-ethylisothiouronium diethylphosphate, and at least one ingredient selected from the group consisting of a buffer, a suspending agent, an isotonizing agent, a preservative, a chelating agent, and aromatic oil.

According to an exemplary embodiment, the intranasal pharmaceutical composition comprises about 0.5% (w/w) to about 5% (w/w) S-ethylisothiouronium diethylphosphate, phosphate buffer, polysorbate 80, polyethylene glycol, benzalkonium chloride, ethylenediaminetetraacetic acid (EDTA), menthol, peppermint oil, eucalyptus oil, and water.

According to another exemplary embodiment, the intranasal pharmaceutical composition comprises about 1% (w/w) to about 3% (w/w) S-ethylisothiouronium diethylphosphate, phosphate buffer, polysorbate 80, polyethylene glycol, benzalkonium chloride, ethylenediaminetetraacetic acid (EDTA), menthol, peppermint oil, eucalyptus oil, and water.

According to a certain embodiment, the intranasal pharmaceutical composition comprises 2% (w/w) of S-ethylisothiouronium diethylphosphate, 10% (w/w) phosphate buffer 100 mM at a pH 6.5, 1% (w/w) polysorbate 80, 2% (w/w) polyethylene glycol 400, 0.06% (w/w) benzalkonium chloride, 0.1% (w/w) ethylenediaminetetraacetic acid (EDTA), 0.1% (w/w) menthol, 0.03% (w/w) peppermint oil, 0.03 (w/w) eucalyptus oil, and 84.68% (w/w) water.

According to another aspect, the present invention provides a method for treating or alleviating upper respiratory and oral-pharyngeal congestion and symptoms associated therewith comprising administering to a subject in need of such treatment a therapeutically effective amount of a pharmaceutical composition comprising as an active agent a compound of formula I:

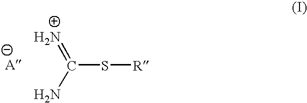

wherein

R″ is a straight or branched alkyl, optionally substituted by halogen; and

A″ (−) is an anion derived from a phosphorous containing acid, a phosphorous acid ester and a phosphorous acid amide.

According to some embodiments, the anion of the compound of formula I is derived from a mono or di-alkyl ester of a phosphate or phosphite.

According to additional embodiments, the compound to be used in the method of the present invention is selected from the group consisting of:
S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

According to a certain embodiment, the compound to be used in the method of the present invention is S-ethylisothiouronium diethylphosphate.

According to additional embodiments, the upper respiratory and oral-pharyngeal congestion is selected from the group consisting of nasal congestion, oral congestion, pharyngeal congestion, and bronchial congestion.

According to further embodiments, the upper respiratory and oral-pharyngeal congestion is due to rhinitis, sinusitis, tonsillitis, otitis media, bronchitis, pharyngitis, laryngitis, surgery, rhinoscopy, and anatomical obstruction of airway passages. According to further embodiments, rhinitis is selected from the group consisting of acute rhinitis, chronic rhinitis, allergic rhinitis, and perennial rhinitis. According to certain embodiments, rhinitis is acute rhinitis or chronic rhinitis.

According to yet further embodiments, the symptoms associated with the upper respiratory and oral-pharyngeal congestion are selected from the group consisting of runny nose, sneezing, difficult breathing, cough, fever, loss of taste and/or smell, headache, throat pain, sinus pain, feeling of pressure or fullness in the sinuses, and dryness of nasal mucosa. According to still further embodiments, the symptoms are selected from the group consisting of hives, swelling, and mild burning of the eyes. According to certain embodiments, the symptoms are runny nose, sneezing, difficulties in breathing including those due to anatomical obstruction of airway passages, and/or dryness of nasal mucosa.

According to some embodiments, the pharmaceutical composition is formulated in a form selected from the group consisting of solutions, suspensions, sprays, creams, gels, ointments, emulsions, tablets, capsules, powders, implants, and sustained-release formulations. According to an exemplary embodiment, the pharmaceutical composition is formulated as a solution. According to certain embodiments, the pharmaceutical composition is formulated as nasal drops or spray. According to some embodiments, if the pharmaceutical composition is formulated as nasal drops or spray, the therapeutically effective amount of S-ethylisothiouronium diethylphosphate is from about 0.5% (w/w) to 5% (w/w) of said pharmaceutical composition.

According to additional embodiments, the pharmaceutical composition further comprises at least one of the ingredients selected from the group consisting of buffers, isotonizing agents, preservatives, pH adjustors, thickeners, chelating agents, suspending agents, perfumes, and natural or synthetic plant extracts.

According to further embodiments, administration of the pharmaceutical composition is performed by intranasal, oral, intravenous, intramuscular, intraperitoneal, transmucosal or transdermal administration route. According to a certain embodiment, administration of the pharmaceutical composition is performed by intranasal administration route.

According to still further embodiments, the pharmaceutical composition is administered prior to onset of the congestion and/or symptoms associated therewith. According to yet further embodiments, the pharmaceutical composition is administered during the congestion and/or symptoms associated therewith. According to still further embodiments, the pharmaceutical composition is administered prior to onset of the congestion and/or symptoms associated therewith and during the congestion and/or symptoms associated therewith.

According to yet further embodiments, the pharmaceutical composition is administered once a day, twice a day, three times a day, four, five, or more times a day so long as the congestion and/or symptoms associated therewith are treated or alleviated. According to a certain embodiment, the pharmaceutical composition is administered three times a day.

According to a further aspect, the present invention provides a method for treating or alleviating upper respiratory congestion comprising administering intranasally to a subject in need of such treatment a therapeutically effective amount of an intranasal pharmaceutical composition comprising as an active agent S-ethylisothiouronium diethylphosphate, the pharmaceutical composition formulated as a solution.

According to yet further aspect, the present invention provides use of a compound of formula I:

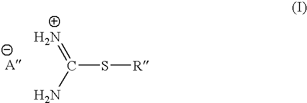

wherein

R″ is a straight or branched alkyl, optionally substituted by halogen; and

A″ (−) is an anion derived from a phosphorous containing acid, a phosphorous acid ester and a phosphorous acid amide, for the manufacture of a medicament for treating or alleviating upper respiratory and oral-pharyngeal congestion.

According to still further aspect, the present invention provides a pharmaceutical composition comprising as an active agent a compound of formula I according to the principles of the present invention for treating or alleviating upper respiratory and oral-pharyngeal congestion.

These and other embodiments of the present invention will be better understood in relation to the description, examples and claims that follow.

### DETAILED DESCRIPTION OF THE INVENTION

The present invention provides compositions and methods for treating upper respiratory and oral pharyngeal congestion and related symptoms in a patient in need thereof.

### Compounds of the Invention

According to the present invention, S-alkylisothiouronium derivative is a compound of formula I:

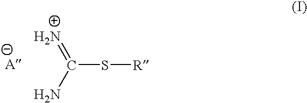

wherein

R″ is a straight or branched alkyl, optionally substituted by halogen; and

A″ (−) is an anion derived from a phosphorous containing acid, a phosphorous acid ester and a phosphorous acid amide.

According to some embodiments, the anion is derived from a mono or di-alkyl ester of a phosphate or phosphite.

According to additional embodiments the compound is selected from the group consisting of:
S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

According to a certain embodiment, the compound is S-ethylisothiouronium diethylphosphate.

### Pharmaceutical Compositions

The pharmaceutical compositions of the present invention comprise an S-alkylisothiouronium derivative and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

The compositions can take the form of solutions, suspensions, emulsion, tablets, capsules, powders, suppositories, implants, sustained-release formulations and the like depending on the route of administration chosen.

The compositions can be in a single dosage form or in a multiple-dosage form.

Routes of administration that are appropriate in the practice of the present invention include intranasal, oral, intramuscular, intraperitoneal, intravenous, intravaginal, intrauterine, rectal, transmucosal and transdermal.

For intranasal administration, the compositions of the invention can be formulated as a solution or suspension, as drops, or as a spray. Preferably, such solutions or suspensions are isotonic relative to nasal secretions and of about the same pH, ranging e.g., from about pH 4.0 to about pH 7.4 or, from pH 6.0 to pH 7.0.

Thus, when the pharmaceutical composition of the present invention is used as nasal drops or spray, it may contain additives such as buffers, isotonizing agents, preservatives, pH adjustors, thickeners, chelating agents, suspending agents, perfumes, natural or synthetic plant extracts such as aromatic oils, etc., which are commonly used in nasal drops unless they are unsuited for the purpose of the present invention.

Examples of buffers include, but are not limited to, phosphate buffers (e.g., sodium dihydrogenphosphate dihydrate, etc.), carbonate buffers (e.g., sodium bicarbonate, etc.), borate buffers (e.g., borax, etc.), citrate buffers (e.g., trisodium citrate dihydrate, etc.), tartrate buffers (e.g., sodium tartrate, etc.), acetate buffers (e.g., sodium acetate, etc.), and amino acids (e.g., sodium glutamate, ε-aminocaproic acid, etc.).

Examples of isotonizing agents include, but are not limited to, saccharides such as sorbitol, glucose and mannitol; polyhydric alcohols such as glycerin, polyethylene glycol and propylene glycol; and salts such as sodium chloride.

Examples of preservatives include, but are not limited to, benzalkonium chloride, benzethonium chloride, parahydroxybenzoates (e.g., methyl parahydroxybenzoate, ethyl parahydroxybenzoate, etc.), benzyl alcohol, sorbic acid or salts thereof, thimerosal, and chlorobutanol.

Examples of pH adjustors include, but are not limited to, hydrochloric acid, acetic acid, phosphoric acid, sodium hydroxide, potassium hydroxide, and borax.

Examples of thickeners include, but are not limited to, hydroxyethylcellulose, hydroxypropylcellulose, methylcellulose, hydroxypropylmethylcellulose, carboxymethylcellulose and salts thereof.

Examples of chelating agents include, but are not limited to, disodium edetate, and ethylenediaminetetraacetic acid (EDTA).

Examples of suspending agents include, but are not limited to, polysorbate 80.

Examples of natural oils include, but are not limited to, peppermint oil, eucalyptus oil, white fir tree oil, wintergreen oil, and menthol.

When the pharmaceutical composition of the present invention is used as nasal drops or spray, examples of the solvents include sterile purified water, distilled water for injection, water for injection, and castor oil.

The compositions of the invention can also be formulated as nasal gels, creams, pastes or ointments. Such formulations may be based upon, simply by way of example, alkylcelluloses and/or other biocompatible carriers of high viscosity well known to the art (see e.g., Remington's Pharmaceutical Sciences, 16th edition, 1980, Ed. By Arthur Osol, the disclosure of which is incorporated by reference herein in its entirety). Other ingredients, such as known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation.

For intranasal administration of solutions or suspensions according to the invention, various devices are available in the art for the generation of drops, droplets and sprays. For example, pharmaceutical composition formulated as solutions can be administered into the nasal passages by means of a simple dropper (or pipette) that includes a glass, plastic or metal dispensing tube from which the contents are expelled drop by drop by means of air pressure provided by a manually powered pump, e.g., a flexible rubber bulb, attached to one end. Alternatively, a spray device can be used for delivery the solution or suspension as a spray.

For administration by inhalation, the compounds for use according to the present invention are conveniently delivered in the form of a droplet, mist or an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

For oral administration, the pharmaceutical composition of the invention can be formulated as tablets, dragees, capsules, liquids, gels, syrups, slurries, suspensions, and the like. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate. Examples of suitable pharmaceutical carriers are described in “Remington's Pharmaceutical Sciences” by E. W. Martin. Such compositions will contain a therapeutically effective amount of an S-alkylisothiouronium derivative together with a suitable amount of a carrier so as to provide the form for proper administration to the subject.

For parenteral administration, the pharmaceutical composition of the invention can be formulated in aqueous solutions, preferably in physiologically compatible buffers such as Hank's solution, Ringer's solution, or physiological saline buffer. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions. Parenteral formulations may optionally contain one or more additional ingredients among which may be mentioned preservatives (when the formulations are presented in multi-dose containers), buffers to provide a suitable pH value for the formulation, and sodium chloride, or glycerin, to render a formulation isotonic with the blood.

For buccal administration, the compositions may take the form of tablets or lozenges formulated in conventional manner.

The pharmaceutical compositions of the present invention can be formulated in an extended release pharmaceutical dosage form as known in the art (see, for example, U.S. Pat. Nos. 6,605,303; 6,419,958; 6,245,357, the content of which is incorporated by reference as if fully set forth herein).

Thus, an extended release pharmaceutical dosage form of the S-alkylisothiouronium derivatives of the present invention comprise an S-alkylisothiouronium derivative, a polymer, and optionally one or more additional pharmaceutically acceptable excipient or carrier.

Polymers that can be used for the preparation of the extended release pharmaceutical dosage form of the present invention include hydrophilic polymers, hydrophobic polymers, and a combination thereof.

Suitable hydrophilic polymers are for instance hydroxypropyl methylcellulose, hydroxypropyl cellulose, ethylhydroxy ethylcellulose, hydroxyethyl cellulose, carboxymethyl cellulose, sodium carboxymethyl cellulose, methyl cellulose, polyethylene oxides, polyvinyl alcohols, tragacanth, and xanthan. These polymers can be used alone or in mixtures with each other.

Hydrophobic polymers are exemplified by for instance polyvinyl chloride, ethyl cellulose, polyvinyl acetate and acrylic acid copolymers, such as Eudragith™. The polymers can be used alone or as mixtures. Alternatively or additionally, hydrophobizing agents can be used for the hydrophobic matrix such as for instance cetanol, cetostearyl alcohol, cetyl palmitate, waxes lice carnauba wax, paraffin, magnesium stearate, sodium stearyl fumarate, and medium- or long-chain glycerol esters alone or in any mixtures.

The extended release pharmaceutical dosage forms of the invention can further comprises binders such as for instance sugars, polyvinyl pyrrolidine, starches and gelatin; surfactants such as non-ionic surfactants such as for instance polysorbate 80, or ionic surfactants such as for instance sodium lauryl sulfate; lubricants such as for instance magnesium stearate, sodium stearyl fumarate, or cetyl palmitate; fillers such as for instance sodium aluminum silicate, lactose, or calcium phosphate; glidants such as for instance talc and aerosol; and antioxidants.

For buccal administration, the compositions may take the form of tablets or lozenges formulated in conventional manner.

Transmucosal or transdermal administration can be accomplished using preparations in the form of ointments, emulsions, gels, lotions, solutions (e.g., douches), creams or patches (in the case of transdermal delivery). Suitable pharmaceutical carriers for transmucosal or transdermal administration include, for example, polyethylene glycol, propylene glycol, glycerin, isopropanol, ethanol, oleic acid, N-methylpyrrolidone, sesame oil, olive oil, wood alcohol ointments, vaseline, and paraffin, or mixtures thereof.

For transmucosal delivery of a gel, cream, or ointment formulation to the vaginal mucosa, bioadhesive polymer-based carrier compositions are particularly useful. Suitable bioadhesive polymers are, e.g., those that are described in U.S. Pat. No. 4,615,697, the content of which is incorporated herein by reference. Particularly useful polymers are cross-linked polycarboxylic acid polymers having a sufficiently high degree of cross-linking to impart the desired level of bioadhesion to the target epithelial surface. Representative bioadhesive polymer formulations are described, for example, in U.S. Pat. No. 5,543,150 and U.S. Pat. No. 5,667,492. Other additives suitable for incorporation into a bioadhesive polymer formulation include one or more of a preservative, a humectant, a lubricating agent and/or moisturizing agent, a stabilizer, a pigment, a pH modifier (e.g., a base) and purified water. Depending on the formulation additives and the resulting viscosity, such formulations may be administered vaginally as a douche, with a plunger, or as a suppository.

Vaginal or rectal suppositories comprising an S-alkylisothiouronium derivative afford constant release over an extended period of time and can be used in the practice of the present invention. Typical base carriers for vaginal or rectal suppositories include, for example, natural, synthetic or partially synthetic fats, waxes and derivative thereof from animal, vegetable, or mineral origin. Specific examples include olive oil, corn oil, castor oil, hydrogenated oils, petrolatum, solid paraffin, liquid paraffin, carnuba wax, bees wax, lanolin, partially or totally synthetic esters of glycerol fatty acid, mono, di, or triglycerides of saturated or unsaturated fatty acids, and well known carriers in the art. Other additives suitable for incorporation into a suppository of the invention include preservatives, stabilizers, surfactants, pigments, pH modifiers and purified water.

The term “about” as used herein refers to ±10% of the numerical value indicated.

### Uses of S-Alkylisothiouronium Derivatives

The present invention provides effective and highly safe methods for treating upper respiratory and oral pharyngeal congestion and related symptoms in a patient in need thereof.

The term “upper respiratory and oral pharyngeal congestion” as used herein includes congestion in the oral, pharyngeal, nasal, and bronchial passages of the upper respiratory tract. Upper respiratory and oral pharyngeal congestion can be due to viral and/or bacterial infections, allergies, colds, poor health, or a predisposition for one or more symptoms through genetic make-up, such as anatomical narrow or obstructed nasal passages or other upper respiratory airway passages, and any other common cause for the congestion.

The term “congestion” as use herein is intended to refer to the narrowing of an airway including the oral, pharyngeal, nasal and bronchial passages due to fluid or a solid substance, such as mucus or phlegm. Narrowing of the airway is often due to swelling or inflammation of the mucous membrane lining the passage to result in a partially or fully blocked passage. Severe cases of congestion often cause difficulties in breathing. However, the present invention encompasses congestion of airway passages due to anatomical structure.

The terms “treating” or “alleviating” as used herein with respect to upper respiratory and oral pharyngeal congestion and related symptoms, include any reduction in severity or duration, of any degree, of the congestion and/or one or more of the related symptoms. The terms also include any delays in onset of and any general relief from the congestion and/or one or more of the related symptoms. Thus, the present invention encompasses palliative compositions and methods.

A wide range of symptoms are related to upper respiratory and oral pharyngeal congestion and depend upon the cause of the congestion. For example, symptoms related to upper respiratory and oral pharyngeal congestion caused by a common cold or flu include, but are not limited to, cough, fever, runny nose, sneezing, difficult breathing, loss of taste and/or smell, headache, and the like. Symptoms related to upper respiratory and oral pharyngeal congestion caused by allergy include, but are not limited to, hives, breakouts, swelling, sneezing, runny nose, mild burning of the eyes, and the like. Symptoms related to upper respiratory and oral pharyngeal congestion caused by viral and/or bacterial infection include, but are not limited to, cough, throat pain, sinus pain, headache, feeling of pressure or fullness in the sinuses, and the like. Besides allergic reactions, infections, and common cold and flu, one or more of the symptoms described herein may also be due to poor health or a predisposition for the symptom through genetic make-up. Thus, the compositions of the present invention can also be used for treating or alleviating difficult breathing in subjects having anatomical obstruction of upper respiratory passages, particularly nasal passages. Also, dryness of the nasal mucosa that causes difficulties in breathing can be treated or alleviated by the compositions of the invention.

The present invention thus provides compositions and methods for treating upper respiratory and oral pharyngeal congestions. Examples of conditions, diseases or disorders that can be treated by the compositions of the present invention include, but not limited to, rhinitis including acute rhinitis, chronic rhinitis, allergic rhinitis, and perennial rhinitis; sinusitis including allergic sinusitis; tonsillitis; otitis media; pharyngitis; laryngitis; bronchitis; nasal obstruction at a post-operative stage; and nasal secretion during rhinoscopy.

The term “therapeutically effective amount” as used herein, is intended to refer to an amount effective for bringing about an improvement in the condition of, and/or relief from, and/or treatment of upper respiratory and oral pharyngeal congestion and/or one or more symptoms related therewith.

The composition may be administered once a day, twice a day, three times, four times a day, or more attain the relief desired, to sustain the relief desired, etc.

Routes of administration that are appropriate in the practice of the present invention include intranasal, oral, intravenous, intramuscular, intraperitoneal, intravaginal, intrauterine, rectal, transmucosal and transdermal administration routes. According to a certain embodiment, the composition of the present invention is administered intranasally.

Combination therapy of an S-alkylisothiouronium derivative and other therapeutically active agents useful in treating or alleviating upper respiratory and oral pharyngeal congestions and symptoms related therewith is also encompassed in the present invention. Thus, S-alkylisothiouronium derivative can be administered before, together, and/or after administration of, for example, a decongestant or an antitussive.

The term “decongestant” as used herein is intended to refer to any agent or ingredient, active for reducing or eliminating congestion of the air passages by widening the airway, and/or by stimulating the release of phlegm and mucus from these passages. Air passages may be widened by reducing the swelling of the mucous membranes in the passage. Generally, sympathomimetic drugs have decongestant properties. Examples of suitable decongestants include, without limitation, phenylethylamine, epinephrine, norepinephrine, dopamine, dobutamine, colterol, ethylnorepinephrine, isoproterenol, isoetharine, metaproterenol, terbutaline, metaraminol, phenylephrine, tyraine, hydroxyamphetamine, ritodrine, prenalterol, methoxyamine, albuterol, amphetamine, methamphetamine, benzphetamine, ephedrine, phenylpropanolamine, mephentermine, phentermine, fenfluramine, propylhexedrine, diethylpropion, phenmetrazine, phendimetrazine, oxymetazoline, xylometazoline, and pseudoephedrine.

The term “antitussive” as used herein is intended to include any agent or active ingredient effective for cough suppression. These include, but are not limited to, common opioid analgesics such as hydrocodone, codeine, morphine, morphine-related compounds including diacetylmorphine, oxymorphone, hydromorphone, dextromethorphan, levorphanol, oxycodone, nalmefene, methadone, meperidine, pentazocine, buprenorphine, nalbuphine, butorphanol, sufentanyl, alfentanyl and propoxyphene, and opioid antagonists not structurally-related to morphine, such as nalorphine, naloxone, naltrexone and fentanyl.

Administration of the pharmaceutical composition of the invention can be performed prior to onset of upper respiratory tract and oral pharyngeal congestion or symptoms associated therewith, at commencement of the congestion or symptoms associated therewith, or both.

The exact formulation, route of administration and dosage can be chosen by the individual physician in view of the patient's condition. (See e.g., Fingl, et al., 1975, in “The Pharmacological Basis of Therapeutics”, Ch. 1 p. 1).

The dosage of the composition to be administered will be dependent on the subject being treated, for example, weight, age, and prior medical history, the severity of the disorder to be treated, the route of administration, the judgment of the prescribing physician, etc.

### Example 1

### Preparation of a Pharmaceutical Composition of S-Ethylisothiouronium Diethylphosphate for Intranasal Administration

The pharmaceutical composition was prepared as follows:
1. Weighing and measuring the active agent and auxiliary substances (S-ethylisothiouronium diethylphosphate, PEG, Nipagin, Nipasol, oils, distilled water for injection).
2. Dissolving Nipagin, Nipasol and volatile oils in PEG (solution #1);
3. Dissolving -ethylisothiouronium diethylphosphate in distilled water for injection (solution #2);
4. Mixing solution #1 with solution #2;
5. Adding phosphate buffer, pH 4;
6. Filtering the solution through 0.2 μm nylon filter; and
7. Dividing the solution in vials.

The composition thus prepared included the following components:

S-ethylisothiouronium diethylphosphate—10 g; peppermint oil (Oleum Mentolum) obtained from peppermint leaves or by synthetic methods—0.4 g; eucalyptus oil (Oleum Eucalypti) having density of 0.91-0.93—1 ml; polyethylene Glycol (PEG)—50 ml; Nipagin—0.2 g; Nipasol—0.4 g; white fir tree oil—1 ml, phosphate buffer pH 4—50 ml; and distilled water up to 1000 ml.

The solution was transparent and colorless, with a slight smell of peppermint, eucalyptus and fir tree oil.

### Example 2

### Effect of Nasal Formulation of S-Ethylisothiouronium Diethylphosphate on Nasal Congestion

Forty patients at the age of 1-20 years suffering from acute sinusitis, acute rhinitis, and atrophic rhinitis were examined. Twenty patients were treated with a 1% solution of S-ethylisothiouronium bromide, designated Olysin, in combination with antibacterial and vitamin preparations (control group). The other 20 patients received the composition listed herein above in Example 1 (treatment group). Each of the patients received one drop of each solution in each nostril, 3-4 times per day.

Analysis of the clinical symptoms in the patients of the treatment group indicated that the preparation had a positive clinical effect, i.e., improved nasal respiration and abolished the pathological secretions. Moreover, the recovery in this group of patients was achieved 2-3 days earlier than in the patients of the control group.

### Example 3

### Effect of Nasal Formulation of S-Ethylisothiouronium Diethylphosphate on Nasal Congestion

A pharmaceutical composition for nasal administration was prepared as follows:

| Ingredient | Amount (% w/w) |
|---|---|
| S-ethylisothiouronium diethylphosphate | 2 |
| Phosphate buffer 100 mM pH 6.5 * | 10 |
| Tween 80 | 1 |
| PEG 400 | 2 |
| Benzalkonium chloride (BKC) 50% solution | 0.06 |
| EDTA | 0.1 |
| Menthol | 0.1 |
| Peppermint oil | 0.03 |
| Eucalyptus oil | 0.03 |
| Water | QS to 100% |

* Buffer composition: Disodium phosphate heptahydrate 0.8% and monosodium phosphate dihydrate 1.08%, filtered through 0.2 μm nylon filter.

A group of 40 patients at the age of 18 to 60 years old, of which 25 were females and 15 men, having the following pathologies: acute sinusitis 12 patients, acute otitis—6 patients, acute rhinitis—10 patients, chronic rhinitis—12 patients, were enrolled to this experiment.

The results of this study demonstrated that intranasal administration of the composition listed herein above brought about a decongestant effect, i.e., disappearance of the sensation of nasal obstruction as reported by the patients. The decongestant effect occurred 2-3 min after the intranasal administration and persisted for 6 hours. Repeated administration was required 6-8 hours after the first application.

The results of these studies indicate that administration of nasal drops of a pharmaceutical composition containing S-ethylisothiouronium diethylphosphate is highly efficient in the treatment of maxillary sinusitis, chronic and acute rhinitis, and of acute otitis, and contributes to the diminishing of nasal obstruction manifested by difficult nasal respiration. The preparation was found to be well tolerated, causing no complications and allergic reactions.

### Example 4

### Effect of Nasal Formulation of S-Ethylisothiouronium Diethylphosphate on Nasal Congestion

Patient A, 8 years old, was admitted with a diagnosis of rhino-sinusitis. The patient displayed vertigo, headaches, and pathological secretions from the nasal cavity that were frequently associated with obstruction of the nasal cavity and difficult respiration. Clinical examination indicated hyperemia of the nasal mucosa, rhinorrhea, nasal obstruction with narrowing of the lower and medium nasal cornets. The patient was treated with, antibacterial medication, and the sinuses were drained by the introduction of antiseptics. In addition, 2-3 nasal drops of S-ethylisothiouronium diethylphosphate 1% were administered. The patient was treated during 3 days. As a result of the treatment with S-ethylisothiouronium diethylphosphate, an amelioration of the clinical symptoms was achieved 1-2 days earlier than that achieved by the treatment with S-ethylisothiouronium bromide (designated Olysin). Also, the repeated administration of S-ethylisothiouronium diethylphosphate was reduced to 2-3 times per day, and not 4-5 times as was necessary when administering Olysin. No adverse effects or tachyphylaxis were found.

Patient B, aged 42 years, was admitted with a diagnosis of rhinitis and acute sinusitis. The patient complained of headaches, dizziness, and common cold. Rhinoscopy demonstrated hyperemia of the nasal mucosa and narrowing of the lower and medium nasal cornets. The radiological examination confirmed acute sinusitis. The patient was treated by puncture of the sinuses with the introduction of antiseptics, Cefazoline 1.0 administered intramuscularly 2 times per day, Loratidin tablets 10 mg and, locally, nasal drops of S-ethylisothiouronium diethylphosphate 2% were used 3-4 times per day. The treatment with S-ethylisothiouronium diethylphosphate led to an amelioration of the clinical symptoms and hyperemia of the nasal mucosa 1-2 days earlier than that obtained by the treatment with Olysin. No adverse effects occurred.

It will be appreciated by persons skilled in the art that the present invention is not limited by what has been particularly shown and described herein above. Rather the scope of the invention is defined by the claims that follow.

1-29. (canceled)

30. Intranasal pharmaceutical composition comprising as an active agent a compound of formula I: 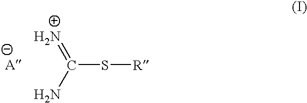
wherein
R″ is a straight or branched alkyl, optionally substituted by halogen; and
A″ (−) is an anion derived from a phosphorous containing acid, a phosphorous acid ester and a phosphorous acid amide; and wherein the compound of formula I is present in the pharmaceutical composition in an amount of from about 0.5% (w/w) to about 5% (w/w), further comprising a pharmaceutically acceptable carrier.

31. The intranasal pharmaceutical composition according to claim 30, wherein the anion is derived from a mono or di-alkyl ester of a phosphate or phosphite.

32. The intranasal pharmaceutical composition according to claim 30, wherein the compound is selected from the group consisting of:
S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

33. The intranasal pharmaceutical composition according to claim 30, wherein the compound is S-ethylisothiouronium diethylphosphate.

34. The intranasal pharmaceutical composition according to claim 30 formulated in a form selected from the group consisting of a solution, suspension, spray, cream, gel, and ointment.

35. The intranasal pharmaceutical composition according to claim 30 formulated as a solution.

36. The intranasal pharmaceutical composition according to claim 30, further comprising at least one ingredient selected from the group consisting of buffers, isotonizing agents, preservatives, pH adjustors, thickeners, chelating agents, suspending agents, perfumes, and natural or synthetic plant extracts

37. The intranasal pharmaceutical composition according to claim 36 comprising about 0.5% (w/w) to about 5% (w/w) S-ethylisothiouronium diethylphosphate, and at least one ingredient selected from the group consisting of a buffer, a suspending agent, an isotonizing agent, a preservative, a chelating agent, and aromatic oil.

38. The intranasal pharmaceutical composition according to claim 36 comprising about 0.5% (w/w) to about 5% (w/w) S-ethylisothiouronium diethylphosphate, phosphate buffer, polysorbate 80, polyethylene glycol, benzalkonium chloride, ethylenediaminetetraacetic acid (EDTA), menthol, peppermint oil, eucalyptus oil, and water.

39. A method for treating or alleviating upper respiratory and oral-pharyngeal congestion and symptoms associated therewith comprising administering to a subject in need of such treatment a therapeutically effective amount of a pharmaceutical composition comprising as an active agent a compound of formula I: 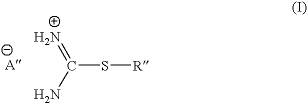
wherein
R″ is a straight or branched alkyl, optionally substituted by halogen; and
A″ (−) is an anion derived from a phosphorous containing acid, a phosphorous acid ester and a phosphorous acid amide.

40. The method according to claim 39, wherein the anion is derived from a mono or di-alkyl ester of a phosphate or phosphite.

41. The method according to claim 39, wherein the compound is selected from the group consisting of:
S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

42. The method according to claim 39, wherein the compound is S-ethylisothiouronium diethylphosphate.

43. The method according to claim 39, wherein the upper respiratory and oral-pharyngeal congestion is selected from the group consisting of nasal congestion, oral congestion, pharyngeal congestion, and bronchial congestion.

44. The method according to claim 39, wherein the symptom is selected from the group consisting of runny nose, sneezing, difficult breathing, cough, fever, loss of taste and/or smell, headache, throat pain, sinus pain, feeling of pressure or fullness in the sinuses, and dryness of nasal mucosa.

45. The method according to claim 39, wherein the pharmaceutical composition is formulated in a form selected from the group consisting of solutions, suspensions, sprays, creams, gels, ointments, emulsions, tablets, capsules, powders, and sustained-release formulations.

46. The method according to claim 39, wherein the pharmaceutical composition is formulated as a solution.

47. The method according to claim 39, wherein the therapeutically effective amount of S-ethylisothiouronium diethylphosphate is from about 0.5% (w/w) to about 5% (w/w) of said pharmaceutical composition.

48. The method according to claim 39, wherein the pharmaceutical composition is administered by intranasal, oral, intravenous, intramuscular, intraperitoneal, transmucosal or transdermal administration.

49. The method according to claim 39, wherein the pharmaceutical composition is administered by intranasal administration.
